(19) United States
(12) Patent Application Publication
(10) Pub. No.: US 2010/0086529 A1
(43) Pub. Date: Apr. 8, 2010

(54) METHODS OF MAKING CONCENTRATED FIBRINOGEN- AND PLATELET-CONTAINING COMPOSITIONS

(76) Inventors: Syed F. Mohammad, Salt Lake City, UT (US); Sivaprasad Sukavaneshvar, Salt Lake City, UT (US)

Correspondence Address: THORPE NORTH & WESTERN, LLP., P.O. Box 1219, SANDY, UT, 84091-1219, US

(21) Appl. No.: 12/576,074

(22) Filed: Oct. 8, 2009

Related U.S. Application Data
(60) Provisional application No. 61/103,751, filed on Oct. 8, 2008.

(51) Int. Cl. A61K 35/16; A61K 38/36; A61K 35/14; C12M 1/00

(52) U.S. Cl. 424/93.73; 514/12; 424/93.7; 435/283.1

(57) ABSTRACT

The present invention is drawn to methods of making concentrated fibrinogen- and platelet-containing compositions. The concentrated compositions can be produced by adding a sufficient amount of a fibrinogen precipitating agent, such as protamine or other similar acting agent(s), and a platelet aggregating agent, such as ADP or other similar acting agents(s), to a platelet/fibrinogen containing fluid to cause the fibrinogen to form a fibrinogen precipitate and the platelets to for platelet aggregations. The fibrinogen precipitate and the platelet aggregates are collected by a collection technique, such as filtration, settling, centrifugation, etc., and are solubilized and deaggregated, respectively, in a liquid vehicle to form a concentrated fibrinogen- and platelet-containing composition. The concentrated platelet and/or fibrinogen compositions can be incorporated into systems for making fibrin glues, hemostatic sealants, platelet rich wound healants, and other wound treating compositions.

This application claims the benefit of U.S. Provisional Patent Application Ser. No. 61/103,751, filed Oct. 8, 2008, which is incorporated herein by reference.

### BACKGROUND

Fibrin-based sealants are frequently used to reduce blood loss during/after surgery. The sealants, formed by mixing a concentrated solution of fibrinogen with thrombin (and optionally Ca²⁺) to produce fibrin, are applied to bleeding wounds and suture lines to help stop bleeding. Concentrated pooled human fibrinogen can be purchased, but it carries the risk of contamination or it is extensively processed to reduce that risk, which adds to the cost of commercial sealants. A method for producing concentrated fibrinogen from autologous blood on short notice would be an attractive alternative to relatively expensive commercial sealants.

The most common method for isolating fibrinogen from human blood is by cryoprecipitation to obtain fibrinogen concentrations of 20-40 mg/mL. This method requires several hours and results in a crude clotting factor concentrate that is useful to manage hemostatically-deficient patients, but is not practical on short notice for small volumes of blood.

Fibrinogen can also be precipitated using chemical agents such as ethanol, polyethylene glycol (PEG), or ammonium sulfate. These methods require somewhat shorter time and provide fibrinogen concentrations ranging from 30 to >50 mg/mL. However, alcohol precipitation can cause elevated levels of ethanol in the fibrinogen concentrate, which can result in premature clotting of the fibrinogen and reduced factor XIII activity (and reduced sealant tensile strength). Often, these methods are also still more time-consuming than would be desirable. Isolation of fibrinogen with ammonium sulfate also precipitates a large amount of albumin, which can interfere with clotting. Precipitation of fibrinogen using PEG requires time-consuming preabsorption of prothrombin using BaSO₄ and MgSO₄, and the presence of PEG or ammonium SO₄ in the fibrinogen preparation is undesirable. Because of these limitations, chemical methods have not been pursued extensively for rapid harvesting of fibrinogen for clinical use as a sealant.

A commercial fibrinogen concentrate, Tisseel VH (Baxter Healthcare Corp., Westlake Village, Calif.), has been available in the United States since 1998. It is prepared by a complex process that includes isolation of fibrinogen from pooled human plasma and heat inactivation or solvent/detergent extraction to reduce the risk of viral contaminants. Tisseel is relatively expensive and has a somewhat limited shelf life. As an alternative to commercial sealants, fibrin sealants have been prepared by mixing plasma or cryoprecipitate with bovine thrombin. However, as mentioned, sealants prepared with lower fibrinogen concentrations as in plasma may not possess desired physicochemical attributes and have limited ability to stop bleeding. Further, the preparation of cryoprecipitates is time-consuming and is generally not cost effective for small volumes. When the plasma or cryoprecipitate are obtained from blood banks, there is also an attendant risk of transmitting blood-borne pathogens.

An attractive clinical approach for augmenting wound healing (or therapeutic treatment in general) is the rapidly expanding clinical and surgical use of recombinant or autologous growth factors for improved therapeutic outcomes. Examples of areas where such wound healing compositions are useful include intractable decubitus and pressure ulcers; orthopedic bone defect repair and bone ingrowth in fixation and implantation procedures; plastic and maxillofacial surgery; burn skin grafts; connective tissue repair; periodontal surgery, etc., as described by: Knighton D R, Surgery, Gynecology & Obstetrics 170: 56-60. 1990; and in Slater M, J Orthop Res 13: 655-663. 1995. Unfortunately, the widespread clinical and surgical acceptance of growth factor-based wound healing therapies are currently limited to some degree by the high cost associated with both recombinant and autologous growth factor healants, and the additional inconvenience of processing autologous cells intraoperatively. Although only few controlled comparisons have been made between autologous growth factor cocktails and purified protein recombinant growth factors for wound healing effectiveness, a single recombinant growth factor may be less effective in many wound healing applications than a combination of growth factors naturally present in platelets as suggested by Cromack D T, J Trauma. 30: S129-S133, 1990. To that end, several potentially therapeutic growth factor compositions have been developed that contain more than one growth factor. However, the clinical applicability of some of these healants can be limited by high cost and inconvenience of obtaining growth factor compositions for use.

### BRIEF DESCRIPTION OF THE DRAWINGS

FIG. 1 is a graphical representation of the recovery of fibrinogen in the form of a concentrated fibrinogen containing composition (percentage of fibrinogen in the original plasma) as a function of the protamine concentration used in the plasma. Data are shown as mean values±SD (n=4).

FIG. 2 is a graphical representation of the recovery of fibrinogen in the concentrate (percentage of fibrinogen in the original plasma) as a function of the precipitation temperature. Data are shown as mean values±SD (n=4).

FIG. 3 is a graphical representation of the Tensile strength (n=4) and adhesion strength (n=8) of a fibrin sealant as a function of calcium chloride concentration. A sealant fibrinogen concentration of 15 mg/mL was used. Data are shown as mean values±SD.

FIG. 4 is a graphical representation of the tensile strength as factor XIII (10 μg/mL) and calcium chloride (8.9 mM) were added to pure fibrinogen (15 mg/mL). Data are shown as mean values±SD (n=4).

FIG. 5 is a graphical representation of the tensile strength (n=4) and adhesion strength (n=8) of sealant as a function of cure time with or without calcium chloride (8.9 mM). The sealant was formed from precipitated plasma fibrinogen (15 mg/mL) at 37° C. and kept at 22° C. for 30 min. Data are shown as mean values±SD.

FIG. 6 is a graphical representation of the tensile strength (n=4) and adhesion strength (n=8) of sealant varied as a near-linear function of fibrinogen concentration and was greater with the addition of CaCl₂ (8.9 mM). Results from clotted Tisseel, plasma, and pure fibrinogen (15 mg/mL) are also shown. Data are shown as mean values±SD.

FIG. 7 is a graphical representation of the tensile strength of 15 mg/mL fibrinogen concentrates prepared from 1) pure fibrinogen, 2) pure fibrinogen precipitated with protamine, centrifuged, and then re-dissolved, and 3) plasma fibrinogen precipitated with protamine, centrifuged, and then re-dissolved. Data are shown as mean values±SD (n=4).

FIG. 8 is a graphical representation of the tensile and adhesion strengths of clots prepared from sealant in the presence of antifibrinolytic agents with and without calcium chloride (8.9 mM). A sealant fibrinogen concentration of 15 mg/mL was used. Antifibrinolytic agents used were aprotinin (3000 KIU/mL) and ε-aminocaproic acid (ε-ACA, 10 mg/mL). Data are shown as mean values±SD (n=4).

FIG. 9 is a schematic representation of a filter design for concentrating fibrinogen from whole blood. The filtration chamber can be designed for a range of blood volumes (e.g. 10-20 mL, 25-50 mL, 50-75 mL, 75-100 mL). The time from adding the blood to the mixing chamber to the recovery of concentrate is usually less than 15 min. The fibrinogen concentrate prepared from whole blood exhibits physicochemical characteristics similar to the commercially available fibrin glue Tisseel V (Baxter Healthcare, Calif.).

FIG. 10 is a perspective view of a device which can be used in conjunction with the methods of the present invention.

FIG. 11 is front view of a mixing/filtering chamber of the system of FIG. 10, and is configured in the mixing position.

FIG. 12 is front view of a mixing/filtering chamber of the system of FIG. 10, and is configured in the separating position.

FIG. 13 is a schematic representation of an alternative embodiment of the device which can be used with the methods of the present invention wherein the mixing and filtering chamber are separated by a valve.

FIG. 14 is a graphical representation showing a hemocytometer count of platelet yield of a recovered cell suspension compared to whole blood and filtrate.

FIG. 15 is a graphical representation comparing function of platelets recovered by the aggregation/filtration process of the present invention and function of platelets recovered using a conventional centrifugation technique of the prior art.

FIG. 16 is a graphical representation comparing PDGF-AB recovery from the aggregation/filtration process of the present invention and PDGF-AB recovery using a conventional centrifugation technique of the prior art.

FIG. 17 is a schematic representation of a hand-held system for collecting platelet aggregates in accordance with an embodiment of the invention.

FIG. 18 is a schematic representation of the hand-held system of FIG. 17 in a partially exploded configuration, with a collection bag thereof shown in a compact, folded condition.

### DETAILED DESCRIPTION

Reference will now be made to the exemplary embodiments and specific language will be used herein to describe the same. It will nevertheless be understood that no limitation of the scope of the invention is thereby intended. Alterations and further modifications of the inventive features illustrated herein, and additional applications of the principles of the inventions as illustrated herein, which would occur to one skilled in the relevant art and having possession of this disclosure, are to be considered within the scope of the invention. It is also to be understood that this invention is not limited to the particular configurations, process steps and materials disclosed herein as these may vary to some degree. Further, it is to be understood that the terminology used herein is used for the purpose of describing particular embodiments only, and is not intended to be limiting as the scope of the present invention.

It is noted that, as used in this specification and the appended claims, singular forms of “a,” “an,” and “the” include plural referents unless the content clearly dictates otherwise.

As used herein, the term “active bleeding” refers to any loss of blood from the circulatory system, regardless of cause.

As used herein, the term “wound” refers to any damage to any tissue of a subject. The wound may, but does not have to be associated with active bleeding. The damage can be injury or surgically created and can be internal or external on the body of the subject. Non-limiting examples of injuries include ulcers, broken bones, puncture wounds, cuts, scrapes, lacerations, surgical incisions, and the like.

As used herein, “fluid” refers to a flowable composition and can include liquid, suspended solids, or other flowable masses. Fluids can be in the form of suspensions, emulsions, solutions, mixtures, colloids, or the like.

As used herein, the term “platelet/fibrinogen containing fluid” refers to any fluid, either biological or artificial, which contains platelet and/or fibrinogen. Non-limiting examples of such fluids include various forms of whole blood and blood plasma.

A “concentrated composition” refers to a fibrinogen or platelet containing composition derived from a platelet and fibrinogen containing fluid, wherein the platelet and/or fibrinogen is present in a medium or liquid that is distinct compared to that of the platelet/fibrinogen containing fluid from which the concentrated fibrinogen is derived. The concentrated composition may, but is not required to, have a concentration of the platelet and/or fibrinogen which is greater than the concentration of the platelet/fibrinogen containing fluid. For example, a concentrated composition can have a platelet and/or fibrinogen concentration which is less than or equivalent to the concentration of platelets and/or fibrinogen in the original platelet/fibrinogen containing liquid, or can be at a concentration which is greater than the platelet and/or fibrinogen concentration of the original platelet/fibrinogen containing liquid. In other words, the term “concentrated” does not infer platelet or fibrinogen concentrations as they relate to the original fibrinogen containing fluid from which the concentrated fibrinogen composition is derived, only that it is concentrated enough to form a clot or aid in the formation of a clot under appropriate conditions.

As used herein, the term “collecting” or “collection” when use with respect fibrinogen precipitate and platelet aggregates refers to the separation of the fibrinogen precipitate and/or platelet aggregates from the bulk of the platelet/fibrinogen containing fluid. Such a step does not require, but does allow for, actual gathering of the precipitate or aggregates. The collection may occur through any number of means in the art including, but not limited to gravity separation, decanting, filtration, and the like.

As used herein, fibrinogen and clotting Factor I are synonymous

As used herein, the term “clotting agent” refers to any fluid or material that facilitates or causes clotting of fibrinogen-containing compositions to form a fibrin glue or sealant. Materials like calcium (e.g., calcium salt), magnesium (e.g. magnesium salt), thromboplastin, actin, thrombin, collagen, platelet suspension, precipitated or denatured proteins, complex carbohydrates, silica, zinc, diatomaceous earth, kaolin, Russel's viper venom, ristocetin, and mixtures thereof, are exemplary. However, clotting agent can also be found in the fluid typically present at a normal wound site, thereby causing the fibrinogen to form a fibrin glue or sealant, though typically at a slower rate.

As used herein, the term “fibrinogen precipitating agent” refers to materials, generally, but not required to be cationic, that react or interact with fibrinogen to cause some amount of precipitation or flocculation, so that the precipitate or flocculent is separable from its fluid to at least some degree. Examples of appropriate fibrinogen precipitating agents include amines such as protamine, polylysine, polyallylamine, histones, and mixtures thereof.

As used herein, the terms “platelet aggregating agent” and “platelet agonists” are used interchangeably and refer to materials which react or interact with platelets to cause aggregation of the platelets, so that the platelet aggregations are separable from its fluid to at least some degree. Non-limiting examples of platelet aggregating agents include collagen, thrombin, ristocetin, arachidonic acid, epinephrine and adenosine di-phosphate (ADP).

As used herein, the term “about” is used to provide flexibility to a numerical range endpoint by providing that a given value may be “a little above” or “a little below” the endpoint. The degree of flexibility of this term can be dictated by the particular variable and would be within the knowledge of those skilled in the art to determine based on experience and the associated description herein.

As used herein, a plurality of components may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0” should be interpreted to include not only the explicitly recited values of about 0.01 to about 2.0, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. This same principle applies to ranges reciting only one numerical value. Furthermore, such an interpretation should apply regardless of the breadth of the range or the characteristics being described.

With these definitions in mind, it has been recognized that it would be advantageous to provide a method of making concentrated compositions of platelets and/or fibrinogen for use in wound healing, particularly the reduction and stoppage of bleeding. The process described herein allows for a cost-effective, timely, and convenient technique for retrieval and concentration of both fibrinogen and platelets which include active growth factors. Platelets may be harvested from small volumes of platelet-rich plasma and/or patient blood preoperatively, intraoperatively, perioperatively, or for outpatient procedures to allow for convenient and sustained delivery of growth factors to effectively promote healing.

The present invention provides for a method of making fibrinogen- and platelet-containing concentrated compositions. The method includes the steps of c) adding a sufficient amount of a fibrinogen precipitating agent to a platelet/fibrinogen containing fluid to cause formation of a fibrinogen precipitate; d) collecting the fibrinogen precipitate; a) adding a sufficient amount of a platelet aggregating agent to the platelet/fibrinogen containing fluid to cause formation of platelet aggregates; b) collecting the platelet aggregates; and after the collection step or steps, e) solubilizing the fibrinogen precipitate and deaggregating the platelet aggregates in at least one liquid vehicle to form at least one concentrated composition.

In one embodiment, the fibrinogen can be precipitated using the precipitating agent and collected prior to the aggregation of the platelets with the platelet aggregating agent and collection of the aggregated platelets. In another embodiment, the platelets can be aggregated with the platelet aggregating agent and collected prior to the precipitating of the fibrinogen with the fibrinogen collecting agent. In yet a further embodiment, the fibrinogen can be precipitated and the platelets aggregated prior to the collecting steps. In other words, the fibrinogen precipitating agent and the platelet aggregating agent can be added to the platelet/fibrinogen containing fluid in the same or sequential step(s) and the precipitated fibrinogen and aggregated platelets can be collected in the same or subsequent sequential step(s).

Once collected the fibrinogen precipitate and the platelet aggregates can be solubilized/resolubilized or deaggregated in liquid vehicle to form one or more concentrated composition(s). In one embodiment, the aggregated platelets and the fibrinogen precipitate can be deaggregated and solubilized, respectively, in separate liquid vehicles thereby forming two separate concentrated compositions, a concentrated composition containing platelets and a concentrated composition containing fibrinogen. In such an embodiment, each composition can be used in wound treatment and/or to aid in the cessation of bleeding together, or combined in a single composition or article. In another embodiment, the fibrinogen precipitate and the platelet aggregates can be solubilized/resolubilzed and/or deaggregated in a single liquid vehicle to form a single concentrated composition containing both fibrinogen and platelets. It is also the case that aggregated platelets or fibrinogen can be used without the need of solubiliizing.

It is noted that when discussing the concentrated compositions, their methods of making, and related methods of use in fibrin glues, fibrin glue systems, platelet-containing compositions, and other therapeutic compositions, each of these discussions can be considered applicable to each of these embodiments, whether or not they are explicitly discussed in the context of that embodiment. Thus, for example, in discussing concentrated composition for use in promoting the stoppage of bleeding, the concentrated composition can also be used in a system or method for making such a concentrated composition, and vice versa.

In accordance with the methods and systems of the present invention, fibrinogen and platelets can be collected from a variety of physiological and artificial fibrinogen containing fluids. In one aspect of the invention, the platelet/fibrinogen containing fluid can be whole blood. In another aspect, the platelet/fibrinogen containing fluid can be plasma, including typical plasma, as well as platelet rich plasma (PRP) or platelet poor plasma (PPP), or even plasma modified with other additives, e.g., Ca²⁺, buffers, or diluents. The source of the blood or plasma can be a human source or other animal source. The present disclosure is particularly useful when it is desired the source of the platelet/fibrinogen containing fluid is also the target for use of the concentrated composition. For example, when prepared in anticipation of surgery or for use in treating an injury. Plasma, platelet, and fibrinogen levels vary considerably between individuals, being affected by age, sex, race, alcohol intake, and smoking, as well as certain diseases. Fibrinogen concentrations of 2-6 mg/mL are typical in normal patient populations; however, clots prepared from unconcentrated fibrinogen solutions can fail to provide desired mechanical properties, thereby leading to poor reproducibility and questionable efficacy as a sealant. The present disclosure provides for the ability to control fibrinogen concentration in the final concentrate, which in turn helps to minimize the variation in sealant performance.

Once a platelet/fibrinogen containing fluid is chosen, a fibrinogen precipitating agent and a platelet aggregating agent can be added to the fluid to cause the fibrinogen to precipitate or flocculate and the platelets to aggregate. There are a variety of fibrinogen precipitating agents which can be used including various amines including protamine, polylysine, polyallylamine, histones, and mixtures thereof. In one embodiment, protamine is the cationic agent. Fibrinogen precipitation by a fibrinogen precipitating agent, such as protamine, is rapid, and often results in much if not substantially all of the fibrinogen in the fibrinogen containing fluid being recovered. It also has the benefit of precipitating certain clotting factors, including Factor X, Factor XIII, and/or Factor II. Alternatively, the fibrinogen can be precipitated by adsorption on a substrate to which a cationic agent or cationic ligand is attached, thereby sequestering the fibrinogen.

The aggregation of the platelets can be accomplished through the addition of the platelet aggregating agent. Platelet aggregating agents can be added to the platelet/fibrinogen containing fluid independently or in conjunction with the addition of the fibrinogen precipitating agent. Non-limiting examples of platelet aggregating agents include thrombin, ristocetin, arachidonic acid, collagen, epinephrine, and ADP. Though all of the above-mentioned platelet aggregating agents are functional, their use in some circumstances may result in the release and loss of granular contents (such as growth factors), or have other adverse processing effects. This aside, certain positive characteristics may outweigh perceived negative effects. For example, collagen may be desired in specific circumstances where growth factors are preferably to be delivered in a collagen substrate. In one embodiment, the use of from 10 μM to 100 μM of ADP can be preferred for platelet aggregation. Both collagen and ADP have some inherent growth promoting features. In another embodiment, the use of collagen or epinephrine can be used for platelet aggregation.

Though the aggregation of the platelets and the precipitation of the fibrinogen may be accomplished at any functional temperature and mixing time, it can be preferable to use certain temperatures to yield certain aggregation results. For example in embodiment the aggregation of the platelets can be done at a temperature of about 15° C. to 42° C., and more preferably at a temperature of about 20° C. to 37° C. Preferred mixing times can be from 15 to 300 seconds, and more preferred mixing times can be from 60 to 180 seconds. If a stir bar is used for mixing in an electromechanical mixer, any functional RPM rate can be used, though from 60 to 3000 RPMs provides a range, and from 200 to 1000 1500 RPMs provides a preferred range for stirring. This speed may vary depending on the geometry of the mixing chamber and geometry of the stir bar. The temperature, time, and stirring force for mixing can be optimized with respect to a specific system, as would be ascertainable by one skilled in the art after reading the present disclosure. In one embodiment, these parameters can be optimized to create platelet aggregates with nominal dimensions of over approximately 15 μm for eventual filtration.

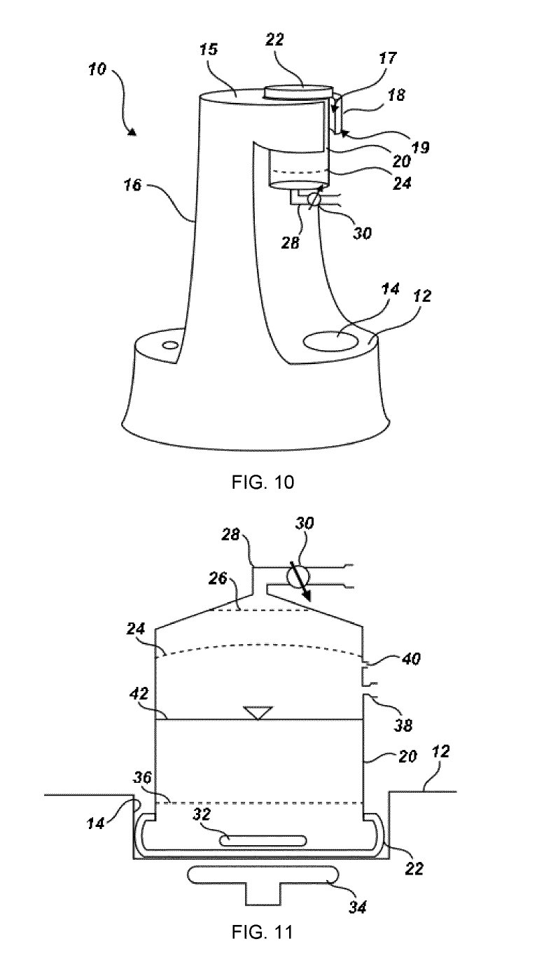

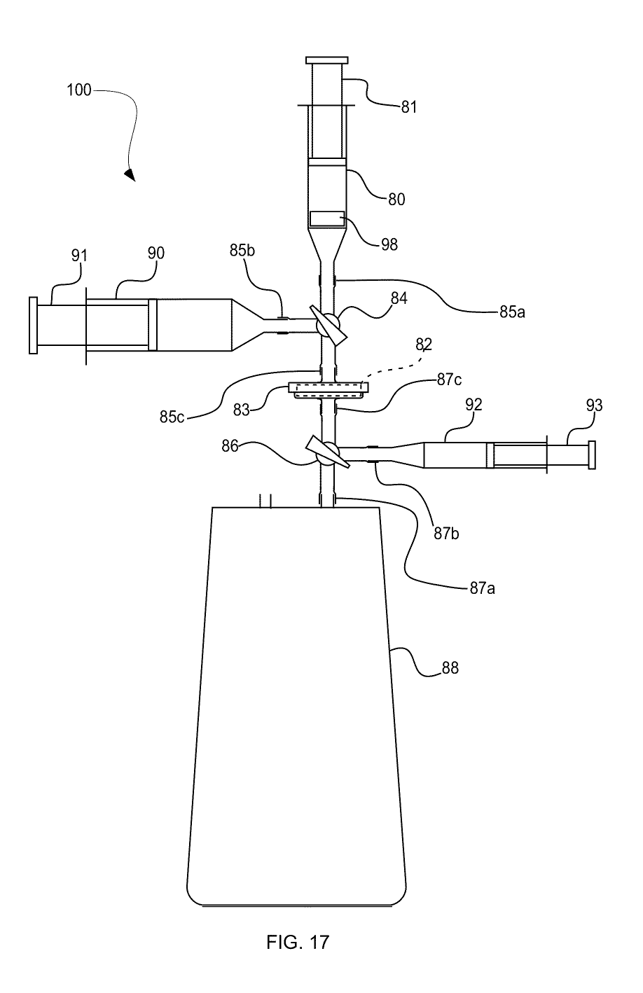

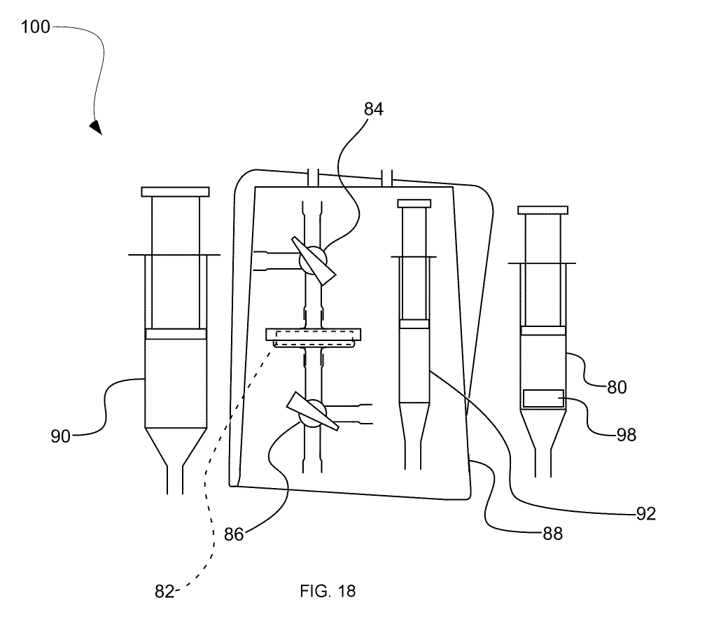

Generally speaking, in some circumstances, the platelet aggregation process should not be allowed to proceed beyond specific time points (typically, 3 minutes or less) dependent upon the aggregating agent utilized. Controlling the elapsed time for aggregation can minimize the risk of growth factor leakage or facilitate the disaggregation. For example, as disclosed in Mohammad S F, Am J Pathol. 79: 81-94. 1975, a lower aggregation time may be preferred by restricting cell aggregation to the first phase of aggregation (for ADP aggregation), and interrupting the process before the second phase of aggregation (the stage during which platelet granular contents are released). Conducting the aggregation process at room temperature, or at temperatures less than 37° C., may also provide some protection against over-aggregation of platelets that could lead to release of granular contents (such as growth factors) that may occur at higher temperatures, e.g., 37° C. However, this could potentially reduce the efficiency of the aggregating process leading to slightly lower yields of platelets compared with aggregation at 37° C. Mixing dynamics of agonist and blood should also be considered for controlled aggregation. If mixing is too gentle, all single platelets may not aggregate. If mixing is too aggressive, high shears and violent collisions may disrupt formed aggregates, thus damaging the cells and releasing the cytoplasmic contents including growth factors. In an electromechanical mixing system as described in conjunction with FIGS. 10-13, optimal ranges of time of aggregation (˜1 to 3 minutes), temperature (˜22° C. to 37° C.), and mixing, e.g. rotational speeds in the order of about 200 to 1500 RPM for proper mixing accomplished by a stir-bar in a cylindrical cup-like chamber, that results in maximum aggregation of platelets and minimum loss of growth factors are desired. In embodiments, such as those describe in FIGS. 17-18, inverting of the mixing chamber to cause a stir bar to pass through the fluid can be repeated as many times as is helpful or desirable to cause adequate mixing. Many times, a stir bar is not required, provided a system of some type is in place to generate mixing of the fluid.

Once the fibrinogen has been precipitated and the platelets aggregated, the precipitated fibrinogen and aggregated platelets can be collected by any collection means known in the art, including but not limited to gravity settling, filtration, or combinations thereof. In one embodiment, the collection is accomplished by filtration. When the collection is accomplished by filtration, the filtration can involve filter assemblies which have single or multiple stages with varying pore sizes, such as about 15 μm and 500 μm, so long as the pore size allows for the retention of the platelet aggregates and the precipitated fibrinogen. In one embodiment, the pore size of the filter can be about 15 μm and 100 μm. Filtration can be advantageous because it can be done using both manual and automated filtration devices. In one embodiment, the filtration device can be as shown in FIGS. 10-12 and discussed in detail below.

As illustrated in FIG. 10, a system, indicated generally at 10, in accordance with an embodiment of the present invention is shown. In accordance with one aspect of the present invention, the system 10 provides a base 12 and a well 14 within the base. Extending upwardly from the base 12 is a longitudinal support 16 terminating in a flat upper surface 15 containing an aperture 17 defined by support 16 and a pair of partially encircling arms 18. Aperture 17 is configured for holding cylindrical mixing/filtering chamber 20.

The cylindrical mixing/filtering chamber 20 has an enclosed expanded end consisting of a flange 22. The flange 22 is larger than the diameter of aperture 17 such that when chamber 20 is inserted into aperture 17 in an inverted filtering or separating position, as shown, the flange 22 will rest on flat surface 15. A space 19 between arms 18 is provided to enable a syringe 44 (not shown) or other device attached at the opposite end of mixing chamber 20 to pass between space 19 when the mixing chamber is inserted into or removed from aperture 17. The well 14 is also configured to hold the mixing chamber 20 at the flanged end 22. Specifically, the flanged end 22 of the mixing/filtering chamber 20 can be placed in the well 14 in a mixing position (not shown). In this position, the mixing/filtering chamber 20 is in position for gently mixing the platelet/fibrinogen containing fluid. Thus, the system 10 provides a means of fixing the mixing/filtering chamber 20 in both a filtering position as shown, and in a mixing position (not shown).

The mixing/filtering chamber 20 is described in greater detail hereinafter. FIG. 10 shows a filter 24 for filtering the precipitated fibrinogen and the aggregated platelets, a stem 28 for removing and adding fluids, and a valve 30 for starting and stopping fluid flow. With respect to the mixing/filtering chamber 20, the outside surface of the cylindrical walls can contain tongues and/or grooves (not shown) and arms 18 can likewise contain matching grooves and/or tongues (not shown) such that, when the mixing device is inserted in aperture 17, it will be locked in a tongue in groove non-rotating position. Likewise, a similar system can be present where the mixing/filtering chamber 20 rests in the well 14.

In FIG. 11, the mixing/filtering chamber 20 is shown in a mixing position. Specifically, the flanged end 22 is shown resting snugly in the well 14 of the base 12. A filter 24, a filter grid 26, and an outlet stem 28 are shown, but are not typically in use when the mixing/filtering chamber is in the mixing position shown. A port 38 is present for transferring platelet/fibrinogen containing fluid, fibrinogen precipitating agent and/or aggregating agent into the mixing/filtering chamber 20. A pressure-reducing vent 40 is also present for allowing air to escape when displaced by the transfer of platelet/fibrinogen containing fluid into the mixing/filtering chamber 20. Both the vent 40 and the port 38 can be equipped with retention members (e.g., stoppers or valves) for preventing the outflow of platelet/fibrinogen containing fluid when the mixing/filtering chamber 20 is in the filtering position. Though only one port and one vent are shown, it is understood that multiple ports and/or vents may be present. For example, separate ports can be used for transferring platelet/fibrinogen containing fluid, fibrinogen precipitating agent, and/or platelet aggregating agent into the mixing/filtering chamber. Alternatively, the platelet/fibrinogen containing fluid, the fibrinogen precipitating agent, and the platelet aggregating agent can be mixed prior to insertion into the mixing/filtering chamber 20. Furthermore, the platelet aggregating agent may be pre-dispensed in the mixing/filtering chamber prior to addition of platelet/fibrinogen containing fluid.

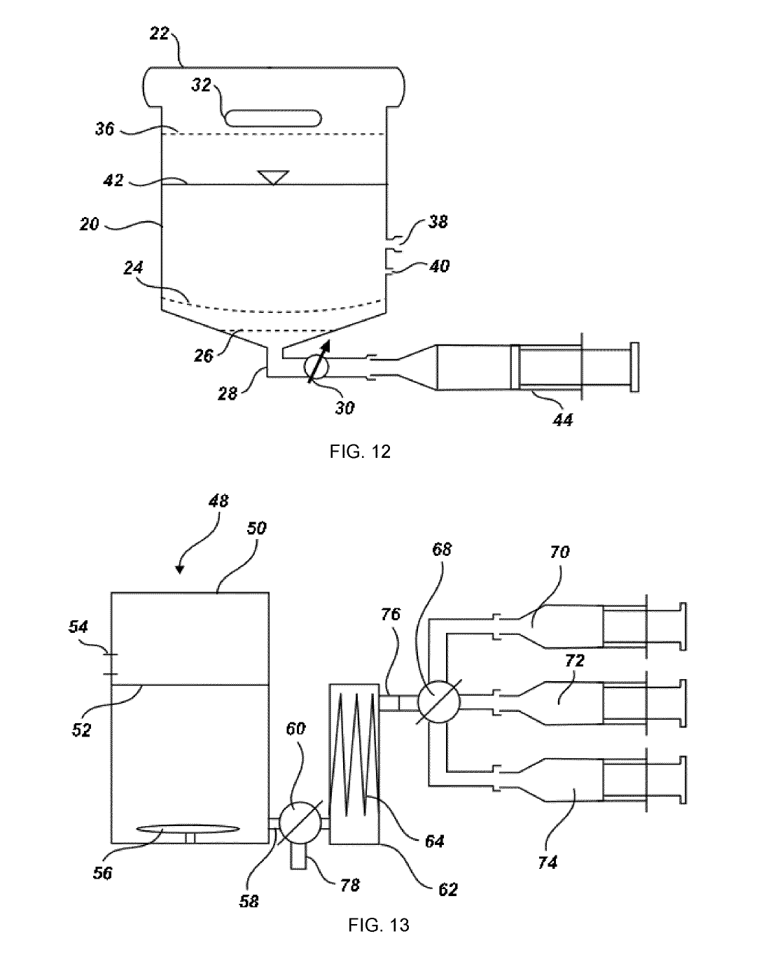

Once the platelet/fibrinogen containing fluid and the platelet aggregating agent and/or fibrinogen precipitating agent are present in the mixing/filtering chamber 20 at a desired fill level 42, a magnetic stir bar 32 can be rotated at flanged end 22 using a motor magnet 34 controlled by a microprocessor (not shown). A Peltier chip for temperature control and timer-alarms can also be present within base 12 or longitudinal support 16, if desired. In the embodiment shown, the motor magnet 34 is located at the bottom of well 14. Any other stirring or mixing configuration can also be used that is gentle enough to mix the platelet/fibrinogen containing fluid with a fibrinogen precipitating agent and/or a platelet aggregating agent without substantially damaging or degranulating platelets, but vigorous enough to thoroughly mix the fibrinogen precipitating agent the platelet aggregating agent with the platelet/fibrinogen containing fluid. A stir bar grid 36 is also present to prevent the stir bar 32 from falling onto the filter 24 when the chamber 20 is inverted as shown in FIG. 12.

Referring now to FIG. 12, the mixing/filtering chamber 20 is shown in a filtering position. The mixing/filtering chamber 20 is held in this position as the chamber is inserted in aperture (not shown) with flanged end 22 resting on flat surface (not shown). The stir bar 32 is prevented from falling into the platelet/fibrinogen containing fluid by the stir bar grid 36.

The filter 24, filter grid 26, stem 28, valve 30, and a syringe 44 are now rendered useful with the mixing/filtering chamber 20 in the position shown in FIG. 12. Specifically, fluid of the platelet/fibrinogen containing fluid is drawn through the filter 24 by creating negative pressure by opening valve 30 and partially withdrawing the plunger of syringe 44. Though a syringe is shown, any pump device can be used. The filter 24 can include one or more filters with nominal pore-sizes ranging from 15 to 500 μm. Further, the filter can be designed to capture the precipitated fibrinogen and/or the aggregated platelets while allowing the passage of non-aggregated cells, e.g. red blood cells and leukocytes, residual aggregating agent, and plasma. The filter can also consist of a removable biodegradable filter, which can be configured to capture fibrinogen precipitate and platelet aggregates, and be applied directly (after washing if desired) to the wound site with little or no further processing.

A filter grid 26 can be present to prevent the filter 24 from getting too close to the stem 28, thus maintaining a larger surface area of filter 24 functional for its intended purpose. The filter 24 has large enough pores to allow non-aggregated blood cells through, but small enough pores to prevent precipitated fibrinogen and/or aggregated platelets from passing. Thus, the precipitated fibrinogen and/or aggregated platelets can be trapped on the filter, and substantially all of the plasma, leukocytes, and red blood cells can be removed.

Referring generally to FIGS. 10 to 12, fibrinogen and/or platelet concentrated compositions prepared according to the methods and systems described herein can be prepared by transferring a desired volume of platelet/fibrinogen containing fluid to the mixing/filtering chamber via an infusion port 38 to attain a desired fill level 42. Inside air can be vented through an air vent 40. A fibrinogen precipitating agent and/or platelet aggregating agent can either be present when the platelet/fibrinogen containing fluid is transferred to the chamber, or can be added to the platelet/fibrinogen containing fluid once in the chamber. The fibrinogen precipitating agent and/or platelet aggregating agent and the platelet/fibrinogen containing fluid can now be manually, semi-automatically, or automatically mixed. It is to be noted that the chamber in which mixing is accomplished is designed to effectively induce platelet aggregation in whole blood without releasing contained growth factors. Thus, mixing should occur that is gentle enough to reduce the release of growth factors, and vigorous enough to promote adequate aggregation. A stable and relatively fixed, rigid, semi-rigid or moldable partially collapsible chamber can be used to reproducibly control mixing patterns and shear rates for whole blood mixing with platelet aggregation agents to achieve appropriate levels of platelet aggregation.

Once adequate mixing has occurred, the platelet aggregates and/or fibrinogen precipitates formed in from the platelet/fibrinogen containing fluid are filtered from the fluid. This is done in the present embodiment simply by inverting the mixing/filtering chamber as shown in FIG. 12. Preferably, the mixing/filtering chamber is also stabilized in the manner previously described. Filtration can occur as gravity forces the fluid of the platelet/fibrinogen containing fluid through the filter 24 and into the stem 28 for removal. However, other methods can be used to cause accelerated flow across the filter as is desired. This flow can be created manually with a syringe, or by connecting to an evacuated chamber, or automatically with a help of a pump or linear actuator. Further, though not required, centrifugation can be used to increase the downward force through the filter and out through the stem. Optionally, a control valve 30 and a filter grid 26 can be used to optimize retention of platelet aggregates and fibrinogen precipitates while effecting removal of other blood components.

Filtered fluid (devoid of aggregates and fibrinogen precipitate) can then be collected in a holding receptacle. For example, a syringe 44 can be used for the holding receptacle. When blood is the platelet/fibrinogen containing fluid the filtered blood can then be returned to the patient, stored (such as for generation of plasma or serum for use as a possible substrate), or disposed of.

An alternative filtration system 48 is shown in FIG. 13. In this embodiment, a mixing chamber 50 is filled with platelet/fibrinogen containing fluid and a fibrinogen precipitating agent and/or a platelet aggregating agent through an inlet port 54 to a desired fill level 52. A mixing mechanism 56, which in this case is a stirring bar, is present for mixing the platelet/fibrinogen containing fluid with the precipitating and/or aggregating agents. A conduit 58 is used to transport the mixed platelet/fibrinogen containing fluid from the mixing chamber 50 to a filtering chamber 62. A valve 60 is present to prevent flow through the conduit in one or both directions when flow is not desired. Once the mixed platelet/fibrinogen containing fluid is in the filtering chamber 62, it is pulled through a porous filter 64 having pore sizes and material properties as previously described, for example. In the present embodiment, the filter is in a pleated arrangement, providing increased surface area if desired. Aggregated platelets and/or fibrinogen precipitates larger than a predetermined size will collect on the filter as residual whole blood components, e.g., plasma, leukocytes, erythrocytes, etc., are allowed to pass.

In the present embodiment, a series of syringes 70, 72, 74 having different purposes are present and attached to a filter port 76 through a valve 68. The valve 68 can be selectively switchable to selectively utilize one of the syringes when desired. If a similar pump system such as provided the series of syringes 70, 72, 74 are desired for use between the mixing chamber 50 and the filter chamber 62, then a valve port 78 can be present as well.

In one embodiment, a first syringe 70 can be used to draw the platelet/fibrinogen containing fluid through the mixing and filtering portions of the system 48. Ultimately, first syringe 70 is used to create the negative pressure desired for flow of the platelet/fibrinogen containing fluid through the system 48. The first syringe 70 is also used to collect fluid from the platelet/fibrinogen containing fluid not collected in the filter 64 as previously described. By turning valve 68 such that fluid communication between the second syringe 72 and the rest of the system 48 can be effected, an aspirating step can occur wherein the precipitated fibrinogen and/or aggregated platelets collected in the filter can be cleaned, such as with saline or another physiological solution, as will be described more fully hereinafter. Still further, the valve 68 can be oriented for functionality of the third syringe 74. The third syringe 74 can be used to inject deaggregating agent into the filtering chamber 62, as will also be described hereinafter. Though the pumping, aspirating, and deaggregating systems shown in this embodiment include a syringe/valve system, other systems could also be used with similar success.

Another embodiment of the invention is illustrated in FIGS. 17 and 18. In this aspect of the invention, a device 100 for separating precipitated fibrinogen and/or aggregated platelets from a fluid suspension is provided. The device can include a mixing chamber 80 that can be operable to receive and mix therein a platelet/fibrinogen containing fluid and a fibrinogen precipitating agent and/or aggregating agent to form fibrinogen precipitate and/or platelet aggregates and fluid from the platelet/fibrinogen containing fluid. A filter 82 (shown enclosed by filter chamber 83) can be in fluid communication with the mixing chamber. The filter can be configured to collect platelet aggregates and fibrinogen precipitate and allow fluid from the platelet/fibrinogen containing fluid to pass there through. A retention member (e.g., valve) 84 can be operably disposed between the mixing chamber and the filter for retaining the platelet/fibrinogen containing fluid and the fibrinogen precipitating agent and/or platelet aggregating agent in the mixing chamber during mixing, and for allowing flow of the fibrinogen precipitates and/or platelet aggregates and residual blood components from the mixing chamber to the filter for filtering.

The mixing chamber 80 can include an optional mixing bar 98 that can be free to move within the mixing chamber and through the fluid suspension, to at least partially mix various components of the fluid suspension. In one aspect of the invention, the mixing bar is a cylindrical piece of stainless steel having a diameter slightly smaller than an inside diameter of the mixing chamber. Once the fluid suspension is contained within the mixing chamber, the mixing chamber can be inverted one or more times to cause the mixing bar to flow through the fluid suspension to mix components of the fluid suspension. While not so limited, in one aspect of the invention, the mixing chamber can be a conventional syringe, for example, a 12 mL syringe of the type commonly available to healthcare practitioners. As described in more detail below, the mixing chamber will generally include structure that enables fluid to be manually drawn into and extracted from the mixing chamber.

While the embodiment of the mixing bar 98 shown and described herein comprises a stainless steel cylinder, or slug, it is to be understood that the mixing bar can take a variety of shapes and can be formed from a variety of materials suitable to provide a mixing force within the mixing chamber/syringe 80. Suitable shapes for the mixing bar can include, without limitation, cylinders, spheres, rectangular shapes and irregular shapes. Also, combinations of any of the foregoing can be utilized, as well as multiple mixing bars used in combination. In one aspect of the invention, a mixing bar is sized so as to allow the expulsion of fluid from the mixing chamber while being restricted (due its size or shape) from exiting or blocking the outlet port of the mixing chamber.

In addition to including a mixing bar 98, in one aspect of the invention, the process of mixing within the mixing chamber 80 can be accomplished without the use of a bar. For example, the geometry (e.g., internal shape) of the mixing chamber can be selected such that movement of the mixing chamber induces mixing of the contents thereof. In addition, creation of gas bubbles within a fluid contained in the mixing chamber can provide sufficient structure to adequately mix the fluid within the chamber.

The retention member 84 (or valve as shown) can be of a variety of types, including, in one embodiment, a “stopcock” valve having two or more male or female Luer ports 85a, 85b and 85c associated therewith. The Luer ports can be sized to receive the outlets of various syringes and/or chambers and the valve can be operable to allow or block flow of fluid from one or more of the syringes and/or chambers, depending upon the configuration of the valve. In the embodiment shown, the valve is configured to allow flow of fluid through only two ports, while blocking flow through the remaining port. For example, the valve can be switched to allow fluid flow through only ports 85a and 85c and not 85b, or through only ports 85b and 85c and not 85a. In this manner, after the fluid suspension has been sufficiently mixed in the mixing chamber/syringe 80 (typically by inverting the syringe once every second or so for several seconds), the mixing syringe can be coupled to port 85a, and the valve can be switched to allow flow of fluid through ports 85a and 85c. At this point, plunger 81 of the mixing syringe can be depressed, forcing the fluid suspension (which now contains fibrinogen precipitate and/or platelet aggregates and residual whole blood components) through filter 82 contained in filter chamber 83.

As the fibrinogen precipitate and/or platelet aggregates and fluid of the platelet/fibrinogen containing fluid through the filter chamber 83, the fibrinogen precipitate and/or platelet aggregates collect on the filter 82 and fluid from the platelet/fibrinogen containing fluid can pass through a second valve 86 (which should be positioned to allow flow of fluid into a collection chamber 88). The f platelet/fibrinogen containing fluid filtrate collected in the collection chamber can either be disposed of after this step, or can be used in some other manner, as discussed below. After the fibrinogen precipitate and/or platelet aggregates have been collected on the filter, a rinse chamber/syringe 90 can be coupled to port 85b. The rinse syringe can contain a solution suitable to remove residual agonist, plasma proteins, loosely trapped cells, etc., with the precipitated fibrinogen and/or aggregated platelets remaining on the filter. Valve 84 can be positioned to allow flow of fluid through ports 85b and 85c. Plunger 91 of the rinse syringe can then be depressed, causing the recovery solution to flow through the filter and rinse the unwanted material from the filter.

It will be appreciated that above described mixing and filtration devices can be readily portable and can be fully operated by a technician without requiring, or significantly benefiting from, input from an external energy source, e.g., without need for electricity. In this manner, an independent fibrinogen and platelet separation system is provided that can be used in areas remote from hospitals, laboratory settings, etc. As shown in FIG. 18, the above described system can be provided in a compact, easily transported and stored device that can be hand-held and hand-operated by a technician.

As will be appreciated by the above description of the components of the mixing and filtration systems, the various processes used in separating platelets from the fluid suspension can be performed manually by a technician. That is, mixing the platelet/fibrinogen containing fluid with an fibrinogen precipitating agent and/or the platelet aggregating agent in the mixing chamber to form fibrinogen precipitate and/or platelet aggregates can be performed manually, for example, by a technician cyclically inverting the mixing syringe one or more times. The fibrinogen precipitate and/or platelet aggregates can be collected or recovered through manually passed through a filter.

Once collected, the fibrinogen precipitate and/or aggregated platelets can be suspended/solubilized or deaggregated in a liquid vehicle to form a concentrated composition. The solubilization and/or deaggregation of the fibrinogen and platelets can be aided by repeated aspiration of the filter and liquid vehicle. The liquid vehicle can be aqueous or non-aqueous so long as it is physiologically acceptable and does not significantly degrade or denature the fibrinogen or the platelets. Examples of liquid vehicles include but are not limited to aqueous solutions of sodium citrate, sodium hydroxide, sodium chloride, potassium hydroxide, heparin, heparan sulfate, other anionic solutions, mixtures thereof and the like. In one embodiment, the liquid vehicle is an aqueous sodium citrate solution.

The concentrated compositions of the present invention can have fibrinogen concentrations which are at least twice the concentration of the platelet/fibrinogen containing fluid from which the fibrinogen is derived. In other words, the methods of the present invention provide for at least a 100% increase in the fibrinogen concentration from the original platelet/fibrinogen containing fluid to the concentrated fibrinogen composition. In one embodiment, the fibrinogen can be present in the concentrated composition at a concentration of 10 mg/mL to 200 mg/mL. In another embodiment, the fibrinogen can be present in the concentrated composition at a concentration of 20 mg/mL to 100 mg/mL. In another embodiment, the fibrinogen can be present in the concentrated composition at a concentration of 20 mg/ml to 60 mg/ml. In a further embodiment, the fibrinogen can be present in the concentrated composition is least about 15 mg/mL.

An additional benefit of the above described methods of harvesting fibrinogen and platelets can be the simultaneous harvesting of the clotting factors which may be present in the platelet/fibrinogen containing fluid. Such clotting factors can include, but are not limited to, Factor X, Factor IX, Factor XIII, Factor II, Factor VIII, and the like, which are present in the plasma and whole blood. As such, in one embodiment, the concentrated compositions obtained by any of the above described methods can include at least one of Factor IX, Factor X, Factor XIII, Factor II, and Factor VIII. In another embodiment, the concentrated compositions obtained by the above described method can include at least two of Factor X, Factor IX, Factor XIII, Factor II, and Factor VIII. In yet another embodiment, the concentrated compositions obtained by any of the above described methods can include each of Factor X, Factor IX, Factor XIII, and Factor VIII. When the concentrated composition is derived from whole blood or plasma, the at least one clotting factor, e.g. Factor X, Factor II, Factor IX, or Factor XIII, can be present in the concentrated composition at a concentration which is at lease twice the concentration of the clotting factor in the plasma or whole blood, though this is not required. The mere presence of these clotting factors in the concentrated composition can provide a benefit for enhancing clotting function.

The concentrated compositions prepared by any of the methods of the present invention can be used to prepare fibrin sealants or glues or other compositions which can be applied to wounds. Examples of wounds include accidental cuts, punctures, internal bleeding, other injuries, surgical incisions, and the like. Thus, by “wound,” this term does not necessarily imply that the wound is open to the atmosphere, but rather, it is open compared to its normal state. Typically, wounds will be open to the atmosphere, but internal bleeding is also included herein. The concentrated compositions of the present disclosure can be applied to wounds by mixing the concentrated composition with an amount of thrombin or other clotting agent in order to form the fibrin sealant. The fibrin sealant can be applied to the wound quickly forming a clot which reduces or eliminates active bleeding from the wound. In one example, if thrombin is used, it can be present in the fibrin sealant in amounts of 50 units/mL to 500 units/mL of the fibrin sealant.

The fibrin sealants made with the concentrated compositions can also include other compounds which can aid in wound healing and blood clotting, such as any of the clotting factors (discussed above) or clotting agents. In one embodiment, the fibrin sealant can include at least one clotting factor selected from the group of Factor X, Factor XIII, Factor II, Factor VIII and mixtures thereof. When present, the Factor VIII can aid in forming a more viscous sealant with desirable attributes. One benefit of having Factor XIII included in the fibrin sealant is that it ensures that the fibrin sealant is cross-linked and, therefore, less susceptible to fibrinolysis. Factor XIII requires calcium as a cofactor to crosslink fibrin, increase the tensile strength of clots, and diminish their breakdown.

Clotting agents which can be used in the fibrin sealants or glues in combination with the concentrated composition include, but are not limited to, calcium salts, magnesium salts, thromboplastin, actin, thrombin, collagen, platelet suspension, precipitated or denatured proteins, complex carbohydrates, silica, zinc, diatomaceous earth, kaolin, Russel's viper venom, ristocetin, and mixtures thereof. Generally, when clotting agents are used with the concentrated composition to form a fibrin glue, the clotting agent the concentrated composition are mixed immediately before application of the fibrin glue to an wound. The clotting agents can be added to or mixed with the concentrated composition to form fibrin glue. In one embodiment, the clotting agent can be present in a separate or second fluid which is mixed with the concentrated composition (i.e. a first fluid) immediately prior to the desired use time for the fibrin glue. In order to prevent premature formation of clotting, the first solution i.e. the concentrated composition, and the second solution containing the clotting agent can be maintained in separate containers until shortly before use. In one embodiment of the invention, the second solution can be provided by the wound itself in the form of wound fluids.

In another embodiment, the fibrin sealant can include calcium or magnesium. The addition of calcium or magnesium to the concentrated composition can increase the tensile and adhesion strengths of the resulting clot, presumably by acting, at least in part, as a co-factor of Factor XIII in crosslinking fibrin. In some cases, threshold concentrations of calcium magnesium can be required in the fibrin sealant to produce maximum effects (8.9 mM for the tensile strength, 3.6 mM for the adhesion strength—concentrations based on calcium or magnesium present as calcium chloride or magnesium chloride), suggesting that sufficient calcium or magnesium is needed to bind the free anionic components present in the fibrin fluid, e.g. citrate from sodium citrate, before its interaction with Factor XIII. Generally, calcium chloride or magnesium chloride concentrations in the fibrin sealant above 0.05 M do not have positive effects on the tensile strength of the resulting clot, and in some cases the tensile strength of the clot can be lessened. Without being limited by theory, it is believed that such a result is possibly due to an increase in ionic strength and partial precipitation of the fibrinogen, both adversely affecting the integrity of the clot. Generally, it is believed that any physiologically acceptable source of calcium or magnesium can be used including calcium or magnesium salts. In one embodiment, the calcium or magnesium can be present as calcium chloride (CaCl₂) or magnesium chloride (MgCl₂). In one embodiment, the calcium can be present as calcium chloride in the fibrinogen sealant at a concentration of from 1.8 nM to 100 nM calcium chloride. In another embodiment, the calcium can be present as calcium chloride in the fibrinogen sealant at a concentration of from 8.9 nM to 50 nM calcium chloride. In one embodiment, the magnesium can be present as magnesium chloride in the fibrinogen sealant at a concentration of from 1.8 nM to 100 nM magnesium chloride. In another embodiment, the calcium can be present as magnesium chloride in the fibrinogen sealant at a concentration of from 8.9 nM to 50 nM magnesium chloride.

When the fibrinogen sealants made with the concentrated compositions of the present invention are applied to wounds they help cement the gaps by adhering the tissue and stop the bleeding through the formation of clots. In one embodiment, the fibrinogen sealant can stop the bleeding of a subject in less than about 5 minutes. In another embodiment, the fibrinogen sealant can stop the bleeding of a subject in less than about 3 minutes. In yet a further embodiment, the fibrinogen sealant can stop the bleeding of a subject in less than about 1.5 minutes. Further, in another embodiment, the fibrinogen sealant can form a clot in vitro in less than about 5 minutes. In another embodiment, the fibrinogen sealant can form a clot in vitro in less than about 3 minutes. In yet another embodiment, the fibrinogen sealant can form a clot in vitro in less than about 1.5 minutes. In yet further embodiment, the fibrinogen sealant can form a clot in vitro in less than about 30 seconds.

### EXAMPLES

The following example illustrates preferred embodiments of the invention that are presently known. However, other embodiments can be practiced that are also within the scope of the present invention.

### Example 1

### Preparation of Fibrinogen and Platelet Concentrates from Pooled Human Plasma

Fibrinogen is precipitated from pooled human plasma by addition of protamine sulfate (Sigma Chemical Co.). The protamine sulfate is used to prepare a stock solution of 40 mg/mL. The protamine is then added to the plasma (final concentration=10 mg/mL), mixed, and then filtered to remove the fibrinogen precipitate. The fibrinogen precipitate is then dissolved in 0.2 M sodium citrate (37° C., pH 7.4) to form a concentrated fibrinogen composition.

To the plasma filtrate 100 μM of ADP is added to the as a platelet aggregating agent. The mixture is mixed and then filtered to remove the platelet aggregates. The filtered platelet aggregates were then deaggregated in a recovery solution of 1.8% sodium chloride solution to form a concentrated platelet composition.

### Example 2

### Simultaneous Preparation of a Fibrinogen and Platelet Concentrate Composition from Human Plasma

To an amount of pooled human plasma, protamine sulfate and ADP are added. The mixture is then thoroughly mixed to allow for the formation of fibrinogen precipitate and platelet aggregates. After mixing, the mixture is filtered and the filtrate is appropriately disposed of. The fibrinogen precipitate and the platelet aggregates are solubilized and deaggregated, respectively, in a 0.2 M solution of sodium citrate (37° C., pH 7.4) to form a concentrated composition containing fibrinogen and platelets.

### Example 3

### Preparation of Platelet-Poor Plasma from Whole Blood

Blood is collected from healthy adult human donors by venipuncture into sodium citrate (Sigma Chemical Co., St. Louis, Mo.; final concentration 0.38 g/100 mL) according to the principles of the Declaration of Helsinki. The blood is centrifuged for 30 minutes at 1200 g to obtain platelet-poor plasma (PPP). The platelet-poor plasma can be used immediately for the preparation of fibrinogen concentrates or can be stored for use at a later time. When stored the PPP should be stored at −80° C.

### Example 4

### Preparation of Fibrinogen Concentrate from Pooled Human Plasma

Fibrinogen is precipitated from pooled human plasma by addition of protamine sulfate (Sigma Chemical Co.). The protamine sulfate is used to prepare a stock solution of 40 mg/mL. The protamine is then added to the plasma (final concentration=10 mg/mL), mixed, and then centrifuged at 1000 g for 5 min to sediment the precipitate. The plasma is then decanted, and the remaining precipitate is dissolved in 0.2 M sodium citrate (37° C., pH 7.4).

### Example 5

### Determination of Fibrinogen and Factor XIII Concentrations

A concentrated fibrinogen solution is prepared as in Example 3. The fibrinogen and Factor XIII concentrations are evaluated with an enzyme-linked immunosorbent assay (ELISA; AssayPro LLC, Brooklyn, N.Y.). The color intensity of the developed ELISA plates is measured with a Dynex MRX microplate reader (Dynex Technologies, Chantilly, Va.) and compared to a standard curve.

The fibrinogen concentration in the plasma is measured with the Clauss method, where plasma samples are clotted in the presence of excess thrombin in a CoaData 2000 Fibrintimer (Labor GmbH, Hamburg, Germany). The clotting times are recorded, and the fibrinogen concentration is calculated from a standard curve.

The amount of protamine bound with fibrinogen in the concentrate is determined by using ¹²⁵I-protamine. Two mg of protamine are labeled with ¹²⁵Iodine by utilizing IODO-GEN precoated tubes (Product 28601, Pierce, Rockford, Ill.) following their recommended protocol. In the final experiment, 1.0 mg ¹²⁵I-protamine is mixed with 99.0 mg unlabeled protamine and then added to 10 mL plasma. The resulting precipitate is washed three times with water, dissolved in 0.2 M sodium citrate and the amount of radioactivity associated with concentrated composition is measured by gamma counting.

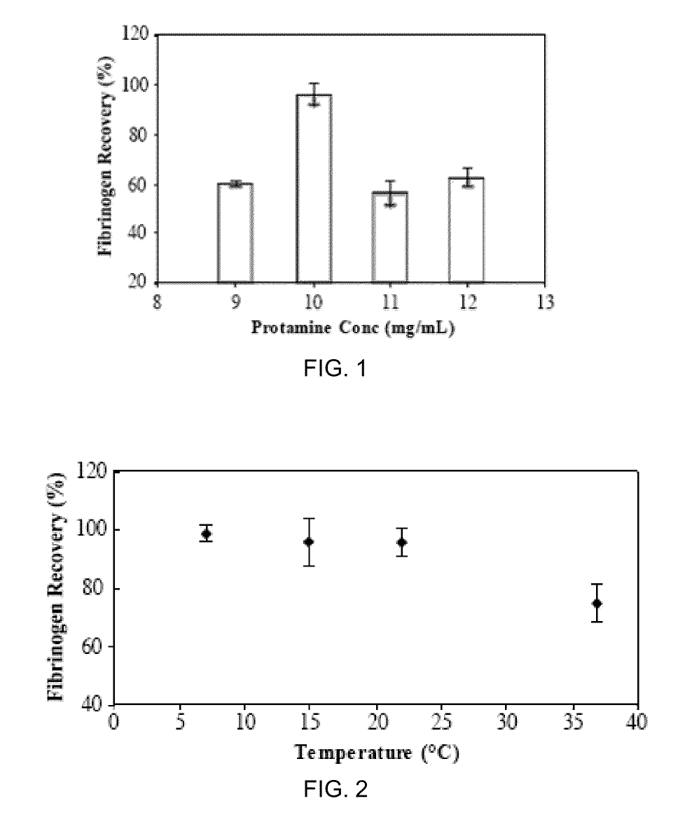

By varying the amount of protamine added to the plasma to achieve final protamine concentrations of 5 mg/mL to 15 mg/mL as guided by the literature various fibrinogen concentrations can be obtained. Maximum fibrinogen can be precipitated and recovered (96±4%, n=4) at a blood or plasma protamine concentration of 10 mg/mL (FIG. 1). Lower protamine concentrations precipitate less fibrinogen, and higher protamine concentrations can result in a precipitate of small dense aggregates that may be difficult to separate and may not readily dissolve.

The extraction efficiency of fibrinogen by using protamine precipitation is affected by temperature. The temperature-dependent nature of the fibrinogen precipitation can be investigated by adding protamine (10 mg/mL) to plasma samples at 37, 22, 15, and 7° C. Fibrinogen recovery is temperature independent at extraction temperatures of 22° C. and lower (FIG. 2) and is significantly better at 22° C. (96±4%, n=4 at 22° C.) than at 37° C. (75±6%, n=4).

The recovery of factor XIII in the concentrated composition when using plasma can reach a final concentration of 3.60±0.05 μg/mL, which is 47±0.6% (n=4) of the factor XIII in the initial plasma.

### Example 6

### Clottability of Precipitated Fibrinogen

The clottability of the recovered fibrinogen is evaluated as follows. A fibrinogen solution as prepared in Example 3 is prepared and used. To 1 mL of the fibrinogen solution 100 μL of bovine thrombin (Vital Products, Inc, Boynton Beach, Fla., 500 Units/mL) is added and the clot is allowed to stand for 30 minutes at 22° C. The clot is then centrifuged for 2 min at 3500 g and the supernatant removed. The amounts of fibrinogen present in the concentrated composition and in the clot supernatant are determined by ELISA, and the fibrinogen present in the clot is determined by difference.

To evaluate the incorporation of Factor XIII in the clot, the above process can be repeated with the addition of calcium chloride (Spectrum Quality Products, Inc., Gardena, Calif.). The amounts of fibrinogen and Factor XIII in the clot supernatant and in the concentrate can be measured with ELISA, and the amounts of fibrinogen and Factor XIII remaining in the clot can be determined by difference.

The fibrinogen in the concentrate polymerizes to form a clot, as described above. The amount of fibrinogen remaining in the clot is determined to be 98±0.9% (n=4) of the amount of fibrinogen in the original concentrate. No change in the clottability of the fibrinogen is observed when calcium chloride is added to the concentrate, and 30±1% of the factor XIII is associated with the clot (n=4).

### Example 7

### Effect of Heparin on Coagulation of a Fibrinogen Containing Concentrated Composition

Heparin is used clinically in most procedures requiring anticoagulation. Heparin is evaluated for its effect on fibrinogen and Factor XIII harvesting and subsequent clotting of harvested fibrinogen. Blood is drawn into syringes containing porcine heparin (ESi Pharmaceuticals, Cherry Hill, N.J.; final concentration 2 U/mL) and centrifuged for 30 minutes at 1200 g to obtain PPP. Protamine was added to a known amount of plasma to bring the plasma concentrations to 10, 11, or 12 mg/mL. Fibrinogen concentrate was prepared as previously described above in Example 3. The amounts of fibrinogen and Factor XIII in the concentrate were measured with ELISA.

When the blood is collected into heparin, the maximum yield of fibrinogen occurs at a protamine concentration of 11 mg/mL in plasma (in contrast to 10 mg/mL when no heparin is present), precipitating 95±1% (n=4) of the fibrinogen in the plasma. At this protamine concentration, 31±3% (n=4) of the Factor XIII in the plasma is found in the concentrate. There are no observed changes in the clottability of fibrinogen when the heparin is present.

### Example 8

### Tensile Strength of Fibrin Clots

The tensile strength of fibrin clots is tested. A dog-bone shaped mold is machined in two halves from plexiglass and forms the shape of the clot. Stiff sponges are placed at the ends to allow the clot to form in/around them; the sponges are held in the mold by bolts in removable plexiglass holders with O-ring seals. The clot diameter is 2 mm in the center of the narrow neck and 6.5 mm at the larger ends, the length is 31 mm, and the mold has a total volume of 1.5 mL. The narrow neck provided the weakest point where the clot would break; the force at which the clot breaks serves as an indication of its tensile strength.

Test samples are prepared by simultaneously emptying syringes of fibrinogen and thrombin into a common duct where the mixture entered the mold through the sponge on one end and exited through the sponge on the other end. Care is taken to avoid introduction of air during filling of the chamber. The sponges, with clot material penetrating their pores, provided a method to grip the clot firmly during testing. After the sample is given time to “cure,” (30 minutes unless various cure times were being tested), the plexiglass mold is dissembled, and the clot is transferred to an Instron Model 1120 Universal Testing Instrument (Instron Corp., Norwood, Mass., max load 500 g) where it is held on the ends via the sponge “grips”. A stress-strain curve is recorded while the sample is strained at 100 mm/min until it ruptured. The tensile strength is recorded as the maximum stress sustained.

### Example 9

### Adhesion Strength of Fibrin Clots

The adhesive strength of fibrin clots is tested. The adhesion strength of the fibrin glue is assessed by sandwiching the fibrin glue between two strips of aortic tissue and then pulling them apart, simulating the performance of the sealant bonding to tissue. Bovine aorta is prepared by slitting the aorta lengthwise and laying it flat. The aorta is then cut into smaller strips, each approximately 3 cm long and 1 cm wide. Since clots do not adhere to the endothelial lining, each strip is cut lengthwise between the adventitia and intima, yielding two thinner strips each with exposed media on one side. Sealant is applied (0.1 mL), covering an area of approximately 1 cm², to the exposed media as shown. An overlapping joint is formed (approximately one-third the length of each strip) and allowed to “cure” while held in place with a 100 g weight for 30 minutes at 22° C. The non-overlapping ends of the cured samples are clamped in an Instron Model 1120 Universal Testing Instrument (max load 500 g), and a stress-strain curve is recorded while the sample is strained at 100 mm/min until the overlapping (glued) joint failed. Adhesion strength is taken as the maximum stress sustained divided by the joint area (indicated by the glue still visible after the joint failed and measured with a digital caliper).

### Example 10

### Effect of Calcium on Tensile and Adhesion Strength

To assess whether the increase in tensile strength when calcium chloride was added to the fibrinogen concentrate (see the Results section) is due to Factor XIII, the tensile strengths of samples prepared from pure fibrinogen (Enzyme Research Laboratories, Swansea, Mid Glamorgan, UK) with and without added Factor XIII (Enzyme Research Laboratories, Swansea, Mid Glamorgan, UK, average functionality of 6200 Loewy units/mg) and calcium are measured. Samples are prepared from a 15 mg/mL pure fibrinogen concentrate (as prepared in Example 4) as follows:

1. fibrinogen alone

2. fibrinogen+calcium chloride (8.9 mM)

3. fibrinogen+factor XIII (10 μg/mL)

4. fibrinogen+factor XIII (10 μg/mL)+calcium chloride (8.9 mM)

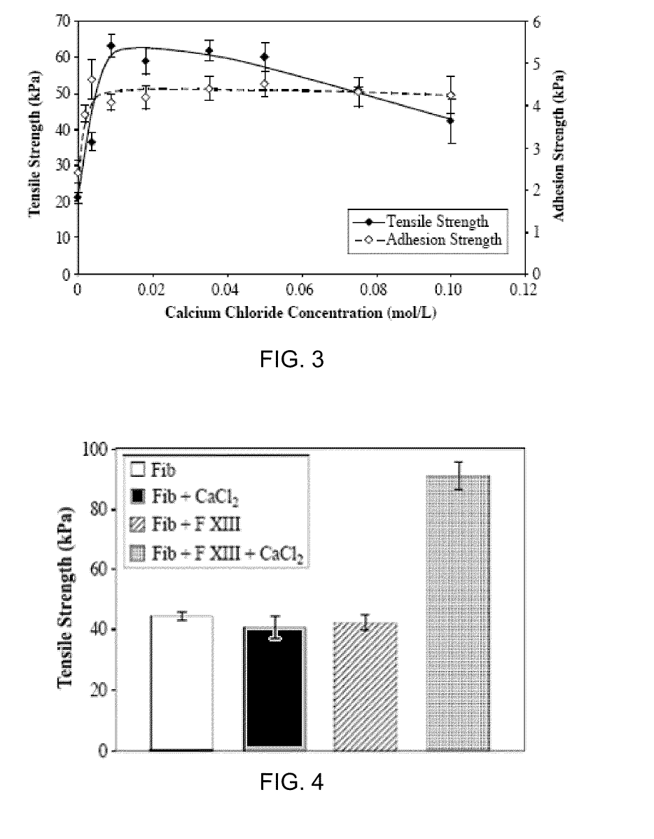

The effect of calcium on clot tensile strength and adhesion strength was investigated by adding calcium chloride (concentrations of 1.8 to 100 mM) to 15 mg/mL fibrinogen concentrate. Maximum tensile strength was achieved with calcium concentrations in the range of 8.9-50 mM, and maximum adhesion strength was obtained with calcium concentrations of 3.6-100 mM (FIG. 3).

Clots were prepared from pure fibrinogen with and without calcium and Factor XIII addition as described above. When Factor XIII and calcium were added together, the tensile strength of the clots increased approximately 50 kPa (FIG. 4), which is similar to the increase of 65 kPa seen in the tensile strength of sealant when the calcium concentration was increased from 0 to 8.9 mM (FIG. 3).

### Example 11

### Effect of Cure Time on Tensile Strength

The effect of cure time on tensile strength and adhesion strength is evaluated by allowing the molded clots and the glued aortic strips (described in Example 9) to cure for 1, 5, 10, 15, 30, and 60 minutes at 22° C. Samples are prepared from a 15 mg/mL fibrinogen concentrate with and without calcium chloride added (8.9 mM).

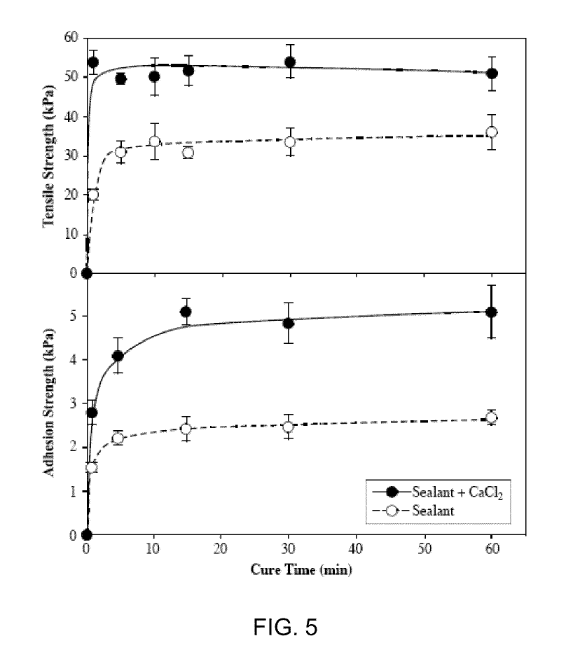

For clots cured for various times, maximum tensile strength was reached in 1 minute (the shortest time that could be measured) with calcium added and about 5 minutes without calcium added (FIG. 5). The maximum adhesion strength with calcium present was approximately twice the adhesion strength without calcium but required a longer cure time to achieve (15 minutes versus 5 minutes).

### Example 12

### Effect of Fibrinogen Concentration on Tensile Strength

To evaluate the effect of fibrinogen concentration on tensile strength and adhesion strength, samples are prepared with fibrinogen concentrations of 15, 30, 45, and 60 mg/mL, with and without calcium chloride added (final concentration 8.9 mM). Controls of pooled human plasma (fibrinogen concentration ˜3 mg/mL), pure fibrinogen (15 mg/mL), and Tisseel (average fibrinogen concentration ˜95 mg/mL) are used. Molded clots and adhesive joints were cured for 30 min.

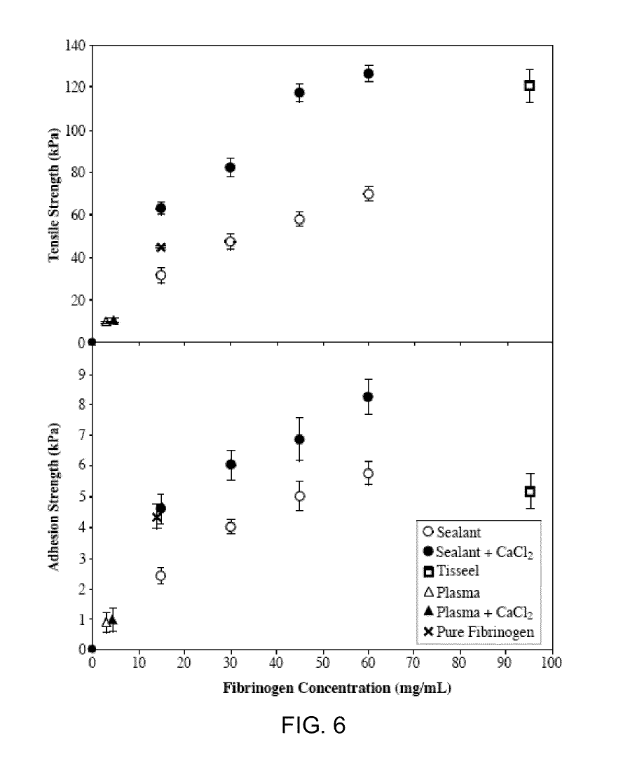

Tensile and adhesion strengths were found to increase approximately linearly with increasing fibrinogen concentration (FIG. 6). The adhesion strength of the sample prepared from plasma fell near the curve with the samples prepared from protamine-fibrinogen concentrate. The tensile strength of the 15 mg/mL pure fibrinogen sample was significantly greater than that of the 15 mg/mL protamine-fibrinogen sample (p<0.05). It appeared that the presence of protamine in the fibrinogen concentrate lowered the adhesion strength of the resulting glue as compared with glue formed with concentrated fibrinogen.

At each fibrinogen concentration, the addition of calcium chloride significantly increased the tensile strength (p<0.05) and adhesion strength (p<0.05) compared with the fibrinogen concentrate with no calcium added. No change in tensile strength or adhesion strength was observed when calcium chloride was added to pure fibrinogen, presumably because there was no Factor XIII present in the pure fibrinogen concentrate. Also, no change in tensile or adhesion strength was observed when calcium chloride was added to citrated plasma. This may have been because either 1) some free calcium was still present in the citrated plasma, thus enabling the Factor XIII action, even when no calcium chloride was added, or 2) the Factor XIII concentration in the plasma was low (normally 10 μg/mL in plasma compared with 20, 50, 70, and 95 μg/mL in the protamine-fibrinogen concentrates).

Tisseel exhibited tensile strength similar to that of sealant made from protamine-fibrinogen concentrate (45-60 mg/mL fibrinogen) with calcium added and adhesion strength similar to that of sealant made from protamine-fibrinogen concentrate (45-60 mg/mL fibrinogen) with no calcium added. The Tisseel adhesion strength was significantly less than that of the sealant glue formed with 30, 45 and 60 mg/mL fibrinogen concentrates with calcium chloride added (p<0.05).

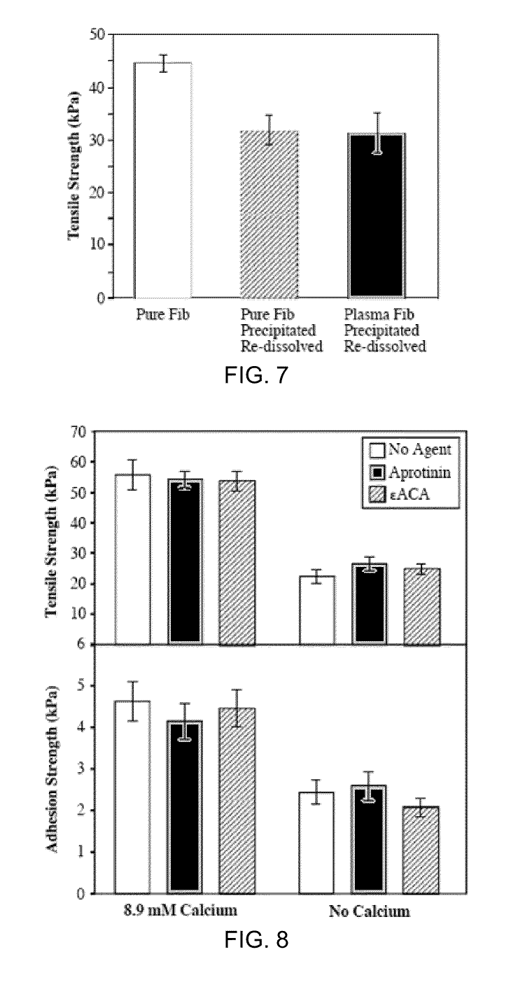

The tensile and adhesion strengths of the 15 mg/mL pure fibrinogen sample were significantly higher than those of the 15 mg/mL protamine-fibrinogen sample (p<0.05). The major difference between these two preparations is the precipitation with protamine in one case. To test the hypothesis that the addition of protamine adversely affected the tensile strength, a fibrinogen concentrate (15 mg/mL) was prepared by protamine precipitation of pure fibrinogen to compare with a 15 mg/mL pure fibrinogen concentrate prepared without precipitation (fibrinogen concentrations were confirmed in both samples). The tensile strength of the protamine-precipitated pure fibrinogen was significantly lower (p<0.05) than that of the pure fibrinogen (FIG. 7), presumably because of the presence of protamine in the concentrate.

### Example 13

### Effect of Fibrinolytic Inhibitors on Tensile Strength

Because enzymes responsible for fibrinolysis in the plasma may affect the clot tensile and adhesion strengths, the effect of the presence of fibrinolytic inhibitors on tensile and adhesion strengths was investigated. Samples were prepared from a 15 mg/mL fibrinogen concentrate with or without calcium chloride added (8.9 mM). In some samples, Aprotinin (Trasylol Injection, Bayer Corp., West Haven, Conn.) was added to the fibrinogen concentrate (final concentration=3000 KIU/mL). In other samples, ε-Aminocaproic acid (Sigma Chemical Co.) was added to the fibrinogen concentrate (final concentration=10 mg/mL). There were no significant changes in tensile strength or adhesion strength upon addition of the antifibrinolytic agents (FIG. 8).

### Example 14

### Preparation of Autologous Fibrin Glue from Whole Blood

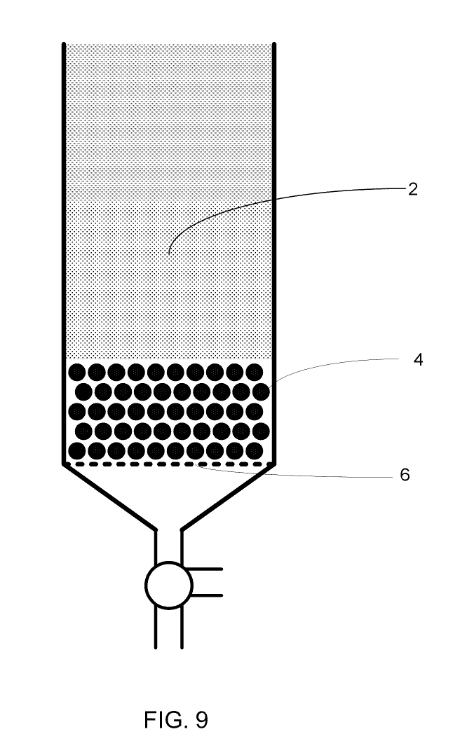

Citrated blood (20 mL) is collected using a blue-top vacutainer system and transferred to a 30 ml syringe predispensed with 200 mg protamine (4.0 mL from a 50 mg/mlL solution), mixed gently for 5 min, and the mixed solution of protamine and blood 2 is poured into a specially-designed tube shown in FIG. 9. The precipitated fibrinogen is captured on a glass-bead 4 (0.1-mm diameter beads in a 1-cm column retained by a nylon mesh filter) as the blood passes through the filter 6. Once all of the blood is drained, the filter is rinsed with three 15-mL aliquots of saline (0.15 M NaCl) to remove nonadherent cells/proteins. After the third rinse, any saline remaining in the tube is drained, the stopcock is closed, and 2.0 mL 0.2M sodium citrate is added. After thorough mixing with a Pasteur pipette, the fluid is drained into a 3-mL syringe as the fibrinogen concentrate. When the fibrinogen concentrate is mixed with a solution of thrombin (500 units/mL of fibrinogen concentrate in 2M CaCl₂; 1:4 vol/vol of concentrate) a viscous fibrin gel forms instantaneously and serves as fibrin sealant. The time from adding the blood to the mixing chamber to the recovery of concentrate is usually less than 15 min. The fibrinogen concentrate prepared from whole blood exhibits physicochemical characteristics similar to the commercially available fibrin glue Tisseel V (Baxter Healthcare, CA).

### Example 15

### Separation of Platelets from Whole Blood

A separation of platelets from whole blood was carried out by the following process: About 10 mL of whole blood was collected from 4 human subjects by venapuncture into syringes having a predispensed anticoagulant contained therein. To the collected whole blood was added 100 μM of ADP as an aggregating agent. The whole blood and aggregate combination was mixed in a chamber with a stir bar for 90 sec at 37° C. Once mixing was stopped, the blood with cellular aggregates was filtered through a filter assembly having pore sizes ranging from 20 μm to 100 μm under negative pressure exerted by a syringe. The filtered aggregates were washed with 30 mL of 18° C. saline for 1 minute. Next, the washed aggregates were incubated with a saline-ACD solution at 37° C. with gentle aspiration for 3-5 minutes. The saline-ACD solution having substantially deaggregated growth-factor containing platelets were then collected as a suspension.

Specifically, to assess the potential effectiveness of the above process of isolation of platelets, the following experiments were conducted: the yield of platelets harvested from human blood was determined and quantified (Example 17); aspects of the functional integrity of the platelets was determined (Example 18); and the presence of one of the most recognized growth factors, PDGF-AB, as a representative growth factor was determined (Example 19).

### Example 16

### Separation of Platelets from Whole Blood

Similar to Example 15, separation of platelets from whole blood can be carried out by the following process: About 10 mL of whole blood was collected from 4 human subjects by venapuncture into syringes having a predispensed anticoagulant contained therein. To the collected whole blood was added 100 μM of ADP as an aggregating agent. The whole blood and aggregating agent combination was mixed in a 12 mL syringe containing a stainless steel mixing bar by inverting the syringe once every 1-2 seconds for 60 seconds, causing the mixing bar to travel through the entire length of the syringe containing whole blood and the aggregating agent. Once mixing was stopped, the plunger of the 12 mL syringe was pushed to force the whole blood containing platelet aggregates through a filter and into a waste collection bag. The filtered aggregates were then washed with 30 mL of 18° C. saline for 1 minute. Next, the washed aggregates were incubated with a recovery solution (saline-ACD solution at 37° C. or 1.8% sodium chloride solution at 18-22° C.) with gentle aspiration for 3-5 minutes. The recovery solution having substantially deaggregated growth-factor containing platelets was then collected as a suspension.

It is noted that Examples 17-20 below relate to the platelet suspension that can be prepared in accordance with Example 3. However, similar results can be achieved using the platelet suspension prepared in accordance with Example 17 or those in other Examples or other similar embodiments.

### Example 17

### Determination of Platelet Yield

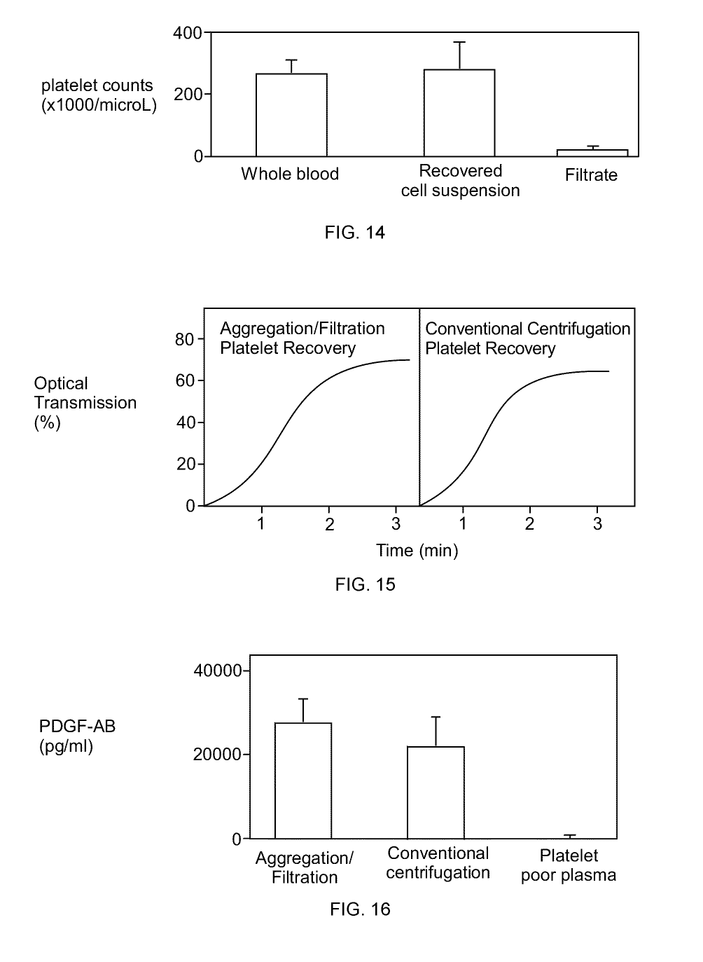

A platelet recovery assay was performed by placing a dilution of a platelet suspension, prepared in accordance with Example 15, in a hemocytometer where the number of platelets were counted using a phase contrast microscope or with the help of an electronic particle counter. Platelets recovered by the present system were compared with platelets recovered using a conventional centrifugation method of the prior art. Hemocytometer counts showed near complete recovery of platelets using the aggregation, filtration, and deaggregation method of the present invention. The results were quantified and are shown in FIG. 14. The waste filtrate from this process contained very few platelets in all cases indicating that aggregation and filtration process was very efficient in harvesting platelets from whole blood.

It is worth noting that two of the four subjects that were part of this study were taking aspirin and/or calcium channel blockers (as anti-hypertension medication). Aspirin is a known suppressor of platelet aggregation, but platelets aggregated well using the methods described in the present invention and good recovery was observed. This being said, some patients with severe platelet deficiencies, or thrombocytopenia, or patients using potent platelet antagonists may not be preferred candidates for this process because their platelet counts may be too low or the functional integrity of their platelets may be compromised. Such candidates may benefit more from platelets collected from a blood donor.

### Example 18

### Determination of Functional Integrity

To assess functional integrity, platelets recovered in accordance with Example 3 were added to autologous platelet poor plasma, incubated for 15 minutes at 37° C., and the function of platelets assessed in a BIO/DATA turbidometric platelet aggregometer using 50 μM of ADP as the aggregating agent. Platelets recovered by the present system were compared with platelets obtained by conventional centrifugation. The comparison of platelet function in a turbidometric aggregometer showed virtually identical platelet aggregation profiles between the platelets recovered by centrifugation, and those recovered by the present invention. FIG. 15 depicts these results. This suggests that the functional integrity of harvested and concentrated platelets obtained by the process of the present invention was not compromised when compared to a prior art method.

### Example 19

### Determination of Presence of PDGF-AB

To determine platelet-derived growth factor (PDGF-AB) presence in platelets concentrated as in Example 15, a chromogenic ELISA method (Quantikine, R&D systems) was utilized. Concentrated platelets obtained by the centrifugation method served as the reference (control) for valid comparisons. Functional viability of the growth factors contained in recovered platelets was assessed by measuring enhancement of human aortic smooth muscle cell proliferation. ELISA results indicated preservation of PDGF-AB in platelets recovered by this process similar to that of PDFG-AB from platelets recovered by centrifugation, as is shown in FIG. 16. This suggests that process steps outlined herein ensure that internal contents of the dense granules in the platelets, e.g., growth factors, are not substantially expelled. The negative control (platelet poor plasma) expressed virtually no PDGF-AB, which suggests valid experimental conditions. The full recovery of PDGF-AB in the platelets harvested by a process of the present invention indicates that other growth factors (PDGF-AA, TGF, VEGF, FGF, etc.) contained in platelets may likewise be preserved during the recovery process.

### Example 20

### Delivery of Platelet Concentrates to Smooth Muscle Cells

The platelet suspension derived by the method of Example 15 enhanced human aortic smooth muscle cell proliferation by 24% compared with the blank buffer control. The results were obtained from two subjects with samples analyzed in triplicate with a MTT assay.

It is to be understood that the above-referenced arrangements are only illustrative of the application for the principles of the present invention. Numerous modifications and alternative arrangements can be devised without departing from the spirit and scope of the present invention while the present invention has been shown in the drawings and fully described above with particularity and detail in connection with what is presently deemed to be the most practical and preferred embodiments(s) of the invention, it will be apparent to those of ordinary skill in the art that numerous modifications can be made without departing from the principles and concepts of the invention as set forth in the claims.

What is claimed is:

1. A method of forming a concentrated fibrinogen and platelet-containing composition, comprising:
a) adding a sufficient amount of a platelet aggregating agent to the platelet/fibrinogen containing fluid to cause formation of platelet aggregates;
b) collecting the platelet aggregates;
c) adding a sufficient amount of a fibrinogen precipitating agent to a platelet/fibrinogen containing fluid to cause formation of a fibrinogen precipitate;
d) collecting the fibrinogen precipitate; and
e) after collecting, deaggregating the platelet aggregates and solubilizing the fibrinogen precipitate in at least one liquid vehicle to form the concentrated fibrinogen- and platelet-containing composition.

2. The method of claim 1, wherein steps a) and b) are performed prior to steps c) and d).

3. The method of claim 1, wherein steps c) and d) are performed prior to steps a) and b).

4. The method of claim 1, wherein steps a) and c) are performed prior to steps b) and d).

5. The method of claim 1, wherein the fibrinogen precipitate and the platelet aggregates are solubilized and deaggregated, respectively, in a single liquid vehicle.

6. The method of claim 1, wherein the fibrinogen precipitate and the platelet aggregates are solubilized and deaggregated, respectively, in two separate liquid vehicles thereby forming two separate concentrated fibrinogen- and platelet-containing compositions

7. The method of claim 1, wherein the concentrated fibrinogen and platelet-containing composition has a concentration which is at least twice that of the fibrinogen and blood platelet concentration in the platelet/fibrinogen containing fluid.

8. The method of claim 1, wherein collecting is performed using gravity settling, centrifugation, or a combination thereof.

9. The method of claim 1, wherein collecting is performed using filtration.

10. The method of claim 9, wherein the filtration is facilitated by suction or pressurization.

11. The method of claim 9, wherein collecting is performed using a portable filtration device.

12. The method of claim 11, wherein the portable filtration device comprises a mixing chamber, a filter configured for fluid communication with the mixing chamber which is configured to collect precipitated fibrinogen and platelet aggregates and allow residual fluid components to pass through, and wherein the mixing chamber and the filter are manually operable by a user without input from an external energy source.

13. The method of claim 1, wherein the collecting of the fibrinogen precipitate and the collecting of the platelet aggregates are each by the same collection technique.

14. The method of claim 1, wherein the collecting of the fibrinogen precipitate and the collecting of the platelet aggregates are each by a different collection technique.

15. The method of claim 1, wherein the liquid vehicle includes a member selected from the group consisting of sodium citrate, sodium hydroxide, sodium chloride, potassium hydroxide, heparin, heparan sulfate, and mixtures thereof.

16. The method of claim 1, wherein the liquid vehicle includes sodium citrate.

17. The method of claim 1, wherein the liquid vehicle includes sodium chloride.

18. The method of claim 1, wherein the liquid vehicle includes sodium chloride and sodium citrate.

19. The method of claim 1, wherein the platelet/fibrinogen containing fluid is whole blood.

20. The method of claim 1, wherein the fibrinogen precipitating agent is selected from the group consisting of protamine, polylysine, polyallylamine, histones, and mixtures thereof.

21. The method of claim 20, wherein the fibrinogen precipitating agent is protamine.

22. The method of claim 1, wherein the platelet/fibrinogen containing fluid is plasma.

23. The method of claim 1, wherein the fibrinogen is present in the concentrated fibrinogen- and platelet-containing composition at a concentration of 10 mg/mL to 200 mg/mL.

24. The method of claim 1, wherein the fibrinogen is present in the concentrated fibrinogen and platelet-containing composition at a concentration of 20 mg/mL to 100 mg/mL.

25. The method of claim 1, wherein the fibrinogen is present in the concentrated fibrinogen and platelet-containing composition at a concentration of from 20 mg/mL to 60 mg/mL.

26. The method of claim 1, wherein the fibrinogen is present in the concentrated fibrinogen and platelet-containing composition at a concentration of at least about 15 mg/mL of fibrinogen.

27. The method of claim 1, wherein the concentrated fibrinogen and platelet-containing composition has a fibrinogen concentration which is at least twice the concentration of fibrinogen in the platelet/fibrinogen containing fluid.

28. The method of claim 1, wherein the concentrated composition further includes at least one clotting factor selected from the group consisting of Factor II, Factor IX, Factor X, and Factor XIII.

29. The method of claim 1, wherein the concentrated composition further includes at least two clotting factors selected from the group consisting of Factor IX, Factor X, Factor XIII, and Factor II.

30. The method of claim 1, wherein the concentrated composition further includes at least three clotting factors selected from the group of Factor II, Factor IX, Factor X, and Factor XIII.

31. The method of claim 1, wherein the concentrated composition further includes each of the clotting factors Factor II, Factor IX, Factor X, and Factor XIII.

32. The method of claim 1, wherein the fibrinogen is precipitated by adsorption on a substrate to which a cationic agent or cationic ligand is attached, sequestering the fibrinogen.

33. A system for forming a concentrated fibrinogen- and platelet- containing composition, comprising:
a) a platelet aggregating agent formulated for aggregating a platelet/fibrinogen containing fluid to cause formation of platelet aggregates; and
b) a fibrinogen precipitating agent formulated for precipitating the platelet/fibrinogen containing fluid to cause formation of a fibrinogen precipitate.

34. The system of claim 33, further comprising a first fluid or device for deaggregating the platelet aggregates, and a second fluid or device for solubilizing the fibrinogen precipitate.

35. The system of claim 34, wherein the first fluid or device and the second fluid or device are the same.

36. The system of claim 34, wherein the first fluid or device and the second fluid or device are different.

37. A concentrated fibrinogen and platelet-containing composition prepared by:
a) adding a sufficient amount of a platelet aggregating agent to the platelet/fibrinogen containing fluid to cause formation of platelet aggregates;
b) collecting the platelet aggregates;
c) adding a sufficient amount of a fibrinogen precipitating agent to a platelet/fibrinogen containing fluid to cause formation of a fibrinogen precipitate;
d) collecting the fibrinogen precipitate; and
e) after collecting, deaggregating the platelet aggregates and solubilizing the fibrinogen precipitate in at least one liquid vehicle to form the concentrated fibrinogen- and platelet-containing composition.
